(19) United States
(12) Patent Application Publication
(10) Pub. No.: US 2020/0257812 A1
(43) Pub. Date: Aug. 13, 2020

(54) Secure Access to Stored Data Files Using Tokens Encoded in Optical Codes

(71) Applicant: Novarad Corporation, American Fork, UT (US)

(72) Inventors: Wendell Arlen Gibby, Mapleton, UT (US); Patrick F. Greenwald, Draper, UT (US)

(21) Appl. No.: 16/273,138

(22) Filed: Feb. 11, 2019

(51) Int. Cl. G06F 21/62; G06F 21/60; G16H 10/60; G16H 30/20; G06F 16/955

(52) U.S. Cl. CPC G06F 21/6209; G06F 21/602; G06F 16/9554; G16H 30/20; G16H 10/60

(57) ABSTRACT

Technology is described for storing and reading a data file using a token encoded in a printed optical code. The method can include receiving the data file over a computer network from a client device. The data file can be sent to be stored in a data store of virtualized data storage accessible via the internet. A token may be received from the data store and the token can be used to access the data file. The data file is returned from the data store when the token is later received from a client device. The token is encoded into an optical code. The optical code can be sent to the client device, where the client device has access to a printer to print the optical code with the token encoded. Any electronic copy of the token can be destroyed.

### BACKGROUND

Computing systems may be found in the workplace, at home, or at school. Computing systems may include computing and data storage systems to process and store data. Some computing systems offer centralized virtualized computing options that may reduce overall costs, improve availability, improve scalability, and reduce time to deploy new applications. For example, some computing systems may act as a service that provides virtual computing, virtual block storage, virtual object storage, virtual networking and other virtual services as purchased for variable periods or on a pay-per-use basis (e.g., pay for a certain amount of API (application program interface) transactions or bandwidth) from large pools of re-purposable, multi-tenant computing resources or services.

Centralized or virtualized data storage systems may be used to store data files of any size and these files may accessible to any computer that can connect to the data storage services over a network connection (e.g., over the internet). Files stored in centralized data storage services enable large numbers of files to be stored and shared between many clients. This type of centralized, scalable storage is often called “cloud” storage. For example, medical records may be stored in cloud storage and then the medical records may be shared on a computer network between medical professionals who are jointly assisting a patient. In another example, video files may be stored in a cloud and accessed by viewers of the videos virtually anywhere in the world.

Massive amounts of digital information are also transferred every day between companies, organizations and individuals using existing computer technologies. This information may be trade secrets, financial accounts, financial data, defense secrets, important product designs, product schedules, or personal photos. While very secure methods have been devised for transferring important or private information, often times such security procedures make the transfer of the data difficult and time consuming. Users of computing systems want to be able to easily, quickly and securely share information, especially those types of information that are sensitive or secret. Systems and method for sharing digital information are desired to be easy to use while also being highly secure in a digital world.

### BRIEF DESCRIPTION OF THE DRAWINGS

FIG. 1 is a block diagram illustrating an example of a system to enable storage of a data file and access to a data file using a token encoded in a printed optical code that can be provided to user.

FIG. 2 is a block diagram illustrating an example of a system for storing an encrypted image file using an encryption key that is printed in a 2D barcode.

FIG. 3 is a block diagram illustrating an example of retrieving a data file using a token, password or encryption key in an optical code.

FIG. 4 is a block diagram illustrating an example of a system to enable access to a medical images or medical data files using a token or encryption key encoded in a printed optical code that can be provided to user.

FIG. 5 is a flowchart illustrating an example of a method for storing and enabling access to a data file using a token encoded in a printed optical code, which can be provided to user.

FIG. 6 is a block diagram illustrating an example of a service provider environment or cloud environment.

FIG. 7 is block diagram illustrating an example of a hardware computing device upon which elements of this technology may be executed.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

When files are stored in a data store or a virtualized data storage service that is centrally accessible via the internet, such files are often the target of malicious attacks. Attackers may wish to obtain a copy of sensitive or valuable files, and so the files may be secured with encryption. However, in the situation where an attacker electronically steals the encryption keys for the files, the attacker can still access centrally stored files. Virtually any data that is connected to a computer network and/or the internet is prone to data hacking or malicious access. Hackers and criminals frequently deduce passwords or repeatedly try to guess password terms in order to steal data, money, individual identity and other digital valuables.

A technology is provided where a token, password, encryption key, login, or similar valuable code is encoded into an optical code (e.g., a 2D optical code) and printed in a physical format (e.g., on paper, plastic, wood, metal, or another medium for printing). The token in the optical code may provide access to a data file stored in a central storage system. In the present technology, if a hacker or individual wants to access information in the associated data file, the optical code must be physically taken from the person who has the optical code because the optical code cannot be accessed through electronic means. Thus, the data file may be securely locked and unlocked using information encoded in the printed optical code.

This technology allows individuals or organizations to store data files in a data store of a data storage service of a public service provider environment (e.g. Amazon, Google, VMWare, Rackspace, etc.) or private cloud storage in a secure configuration. Security may be applied to the data files on a per file basis. Each data file may have a separate token, password or encryption key to encrypt and/or access the data file. A token, password, or encryption key may be encoded into an optical code and printed to enable a user to later access the data file using the printed optical code. A user may later scan in the optical code using a camera or scanner and an application will decode the token, password or encryption key from the optical code. The token, password or encryption key can be sent to the data store which can reply with a readable copy (e.g., decrypted copy) of the data file. Any type of data files may be stored in the data store including images, text, databases, medical images, photos, digital currency, source code, executable code, or other data files.

In this technology, an internet-based system may be used for sending and receiving a data file. For example, images to be stored as data files may come from a medical image capture device in a format that contains a defined image modality (e.g., MRI images, CT scans, ultrasound images, etc.). The image may be sent to a file router, and the file router may send the image to a data store in a cloud-based system. The file router may be a DICOM (Digital Imaging and Communications in Medicine) type of router. There is an authentication that may occur between the file router and the cloud-based data store or archive. The user sending the file may also be authenticated, if desired. The data store or archive can send back a limited use token, which has a secure alpha numeric value (e.g., a 256-bit token, a password or an encryption key). The token may be encoded into an optical code, such as a 2D optical code, QR code, linear barcode or any other type of optical code. The token can then be printed onto a sticker, label, card or a paper and can be provided to a user (e.g., a medical patient).

In one example configuration, the user may use a mobile phone, mobile device, or another client device to scan or read in the QR code using a camera or scanner of the client device. The mobile phone or client device can send the QR code to a local file router or a file service executing in the service provider environment (e.g., Amazon AWS, Microsoft Cloud, Google's cloud) or a private cloud. The file router or file service may decode the token or key from the QR code and send the token or key to the data store.

The data file may then be decrypted by the data store, and the data file may be returned from the data store to the mobile phone or client device. The mobile phone or client device may open a web page for the user (e.g., using a standard HTML5 viewer) or using a web application with the data file where the user can print the data file, email the link to a data file, text the link to the data file, export the data file to a storage device, or load the image file into a compressed file. Marketing information can also be added to any printed page with the printed optical code such as a provider's name (e.g., hospital name), logos, slogans, advertisements, coupons, or other promotional material.

If the token or key is printed, then any electronic versions of a token or key may be deleted. The destruction of electronic copies allows the entity that created and stored the data file to avoid ongoing liability for the security of the data file because the user who received the printed token or key has the sole control of the token or key. Printing the token or encryption key also prevents computerized and/or network access to the token or encryption key until the optical code is scanned back into electronic format. A user that has an encryption key printed in physical form may choose when to scan the 2D optical code back into a computing device using the 2D optical code. The printed token or key is also secure because no other entity has the token, password, or encryption key after the token is printed.

Using a printed token allows a user to carry the token or key with the user or physically store the token until the user desires to access the data file. This is because the printed optical code with the encoded token is small, compact and easy to manage. The printed token can be placed in a user's wallet, on a credit card, on a banking check, or on medical patient card in order to allow a secure data file to be associated with such items.

This physical format also allows the printed 2D optical code to be easily transferred between individuals. This token in the 2D optical code can be very secure because it can also be encrypted using an encryption key with over 800 bits of security in the encryption key.

Further, the user can avoid typing the entire code into a web page, UI (user interface) form, or another interface because the token is embedded into the 2D optical code and may be machine read from the 2D optical code. In addition, another human cannot quickly look at and decipher the optical code to read a token or key from the optical code because a human generally needs a 2D optical code application to read the 2D optical code. Creating a physically printed copy of the token, password or encryption key for centrally accessible data files helps thwart the hacking described earlier.

Some types of data are considered more sensitive than other types of data. Examples of sensitive data include personal identity information, financial data and medical records. For instance, in the medical world, hospitals and other health care providers have a high level of fiduciary duty to protect and care for medical data. Medical data usually contains confidential personal information (birth dates, addresses, SSN, etc.) and can be used for identity theft. Regulators are also strict with medical institutions because there are extensive federal and state regulations that cover the protection of medical records. Public personalities and celebrities also have a strong desire to keep their information private (e.g., medical, psychological, illness, or photo data). In another example, if a user has photo or image that the user wants to keep secure or secret (e.g., top secret documents or a picture of a crime scene), then the token, password or key that is associated with the data file can be encoded into an optical code. The optical code can then be provided to a recipient in a printed format. When the recipient wants to view the photo, then the recipient scans the optical code and is able to obtain access to the photo. The present technology provides a system and method for securing valuable data files on an individual data file basis where the token, password or key for the individual data files is not stored electronically but is readily accessible using a printed physical medium.

While the type of encryption key described above can be made long enough to make the key difficult or time consuming to crack, an individual cannot remember the entire token or key to access a file encrypted with the key. The present technology provides a stronger token or encryption key in a printed format where a user does not need to remember alpha-numeric values of the token or key (or even store the key electronically) in order to access the data file stored with that token or key. In the past, a user may have been provided with a random alpha numeric password that was 6-8 characters in length for accessing a protected file. While a user can remember a password of this length or write the password down, the password is generally weaker because the passwords may be repeated for users of the system and can be more easily attacked by a malicious individual. This technology overcomes this problem of weaker passwords for files by using longer and stronger per file tokens or keys that are stored in a machine readable printed format and does not have to remembered or written down. Remembering or writing down a token, password, or encryption key is also fraught with human error (e.g., perhaps a token is erroneously written down). This technology avoids such errors.

This technology and system can create a fire wall for protected files on a per file basis. Even if a malicious person attacks the data store or data file archive, each data file 116 has an individual token, encryption key or password. If one token or encryption key is cracked or determined by a hacker, the hacker is only able to access one file for one user and none of the other files in the data store 118 (e.g., in the cloud storage) are likely to be compromised. This configuration avoids the situation where a single compromised password or encryption key may allow a hacker to access the data of millions of user's data in a single exploit.

In a further configuration, since there is a separate token, password or encryption key for each data file, then each data file is separately encrypted. If a hacker physically obtains the optical code with the token, password or encryption key, the data files on the data store can be physically decrypted and/or may be accessible. In the situation where a hacker obtains the token or password (e.g., through loss or violence), a different encryption key or scheme may be quickly determined for the data, then the data file may be decrypted and re-encrypted using a new encryption key (i.e., this assumes a data store managed encryption key or backdoor to perform this operation). Thus, if a breach of the printed optical code is known or detected and the password or encryption key to access the data store has been compromised, the encryption key for the data files may quickly be changed and the security of the data files may be maintained. Even in the worst case, if an attacker were able to obtain the decrypted password for one file and/or the decrypted data file itself, only one file would be compromised and not a large number of files (e.g., thousands or millions of files). If this particular password or encryption code happened to give a hacker access to a large volume of data, then a secondary system may track whether a large volume of data is being downloaded in a short period of time, then the download of information could be stopped and the encryption on the data file may be changed.

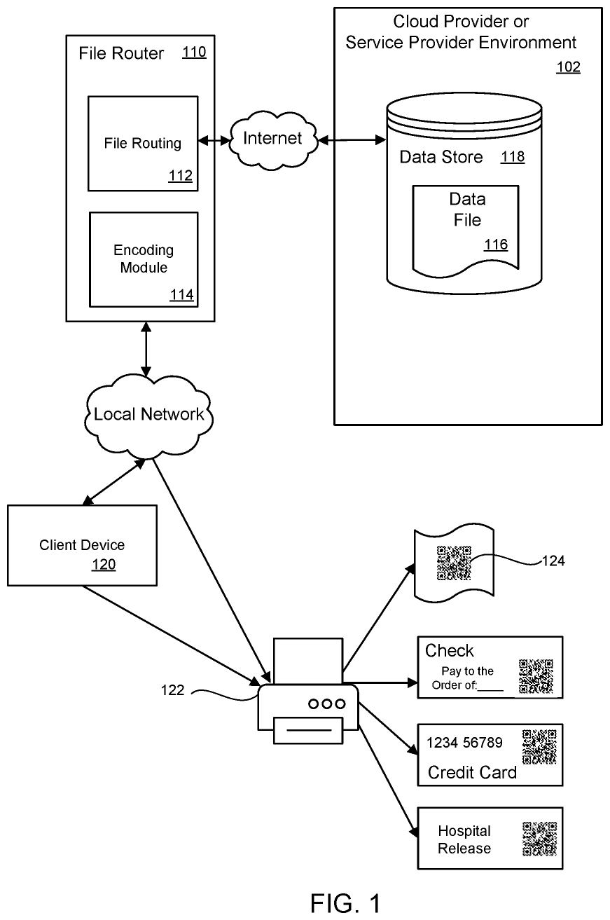

The use of the 2D optical code may also be used to initially setup a medical account, bank account, email account, media account, gaming account or any other web account. The account creator or institution may provide a 2D bar code that includes a token or encryption key to access a data file with an account number, a login, a URL, a password or any other information that can be decrypted from a data file and then can be used to login to an electronic account. For example, the optical code may be used to store a secure login and/or password for logging into a bank website or other website. A bank may provide a user with a physical card with the optical code and encoded token printed on the card. As a result, the printed optical code can be scanned and the secure login name and/or password may be decoded and used to access to the bank website. Alternatively, the printed optical code may have a token, password, or encryption key for a data file stored in the cloud that contains the login and/or password. Thus, the data file can be retrieved and the secure login and password can be used by the user's application to login to the bank's web application. This is much more secure than a regular password. Using an optical code also allows lengthy codes to be embedded in the 2D optical code but a user can easily access the data in the optical code or data in the data file to login to the account by simply scanning the printed 2D optical code. The printed 2D optical code may be read by scanner or optical reader on a credit card reader, a check scanner, a mobile device with a camera (e.g., cell phone or optical code reader), or a computer. FIG. 1 illustrates technology to enable secure access to a protected or encrypted data file using a printed optical code provided to a user. The optical code can have a token, password or encryption key encoded into the optical code. For example, the optical code may be a QR code or another 2D barcode.

A data file 116 may be received at a file router 110 over a computer network from a client device 120. The user of the client device 120 may be making a request to securely store the data file 116. The data file may be a photographic image, a text file, an HTML file, a medical image, a JSON document, a spreadsheet, computer source code, computer object code or another data file 116 that user would like to store and protect.

The data file 116 may be sent by a file routing module 112 of the file router 110 to be stored in a data store 118 of a service provider environment 102 accessible via a computer network, such as the internet. An authentication may also take place between the file router 110 and the data store 118 or between the client device 102 and the data store. The data store 118 may be an object data store (e.g., such as Amazon's S3 or a private object data store), a file storage service, a NoSQL data store or key value data store, a relational data store or another type of data store that can store the data file.

A token may be generated by the data store 118 to be used to access the data file. Where the token is an encryption key, the data file 116 may be encrypted with the encryption key. If the token is a password, then the password may be used in protecting or encrypting the data file. At a later point in time, the token may be presented by a client device 120 to the data store 118, and the data file 116 may be returned to the client device 120 from the data store 118. The token generated by the data store 118 may be sent to the file router 110.

The token received by the file router 110 from the data store 118 may be encoded into an optical code, such as a 2D optical code or QR code. Additional information may also be encrypted and encoded in the optical code such as a web address or URL for accessing a data store. Protecting the location or URL of the data files is important so that hackers do not know the address of the data store where files are located. Unless a hacker has access to the physical copy of the 2D optical code, a hacker will have almost no opportunity to even find out where the files reside. The encoding of the token can take place in the encoding module 114 of the file router 110. Any additional encryption or decryption of passwords or tokens may also take place in the encoding module 114. The file router 110 may be a network router or network access point with additional logic to perform the encoding and file routing.

The optical code may be sent to the client device 120 that was requesting storage of the data store 118. The client device 120 may have access to a printer device 122 to print the optical code with the token, password or encryption key encoded. The optical code with the encoded token or encryption key may be printed in a physical format. For example, the optical code may be a 2D optical code containing the token, such as a QR code or optical code 124 printed on paper or plastic. In alternative configurations, the optical code may be a 2D barcode, a PDF 417 code, an Aztec barcode, or a one-dimensional barcode.

In one configuration, the optical code may be sent directly to the printer device 122. For example, the printer may have a network address and rather than sending the optical code to the client device, the printer device may receive the 2D optical code, QR code, etc. directly from the file router 110 via the computer network. Alternatively, the optical code may be sent directly from the data store to the printer device. The printing of the optical code may contain additional information embedded in a web page with the optical code such as a user data (e.g., a user name or user attributed), a time and place, an entity printing the optical code, etc.

Using the optical codes and the optical scanning avoids using an RFID (Radio Frequency ID) chip which broadcasts a radio frequency signal that can be intercepted by RFID scanners controlled by hackers. Sometime RFID chips can be read through wallets and clothing. In contrast, an optical code cannot be read unless the optical code is presented visibly by a user and is clearly visible to the optical scanner. Similarly, if the optical code is printed and the electronic copes are erased, then when the user physically destroys the optical code (e.g., shreds or burns the printed optical code), the data file may be permanently inaccessible.

The token, password, or encryption key may be time limited and may be set to expire after a set time period has passed. For example, a token may be set to expire after a few hours, after one or more days, after one or more week, after one or more month or after one or more years. Thus, the token may be used to access the data file 116 that was stored in a data store 118 in a service provider environment 102 but if the set time period has passed, then the data file may be inaccessible. More specifically, the data store 118 may reject requests to provide a data file 116 in response to an expired token.

After the file router 110 has sent the token to the client 120, the router may erase any electronic copy of the token. As a result, there may be no electronic copy of the token that might otherwise be electronically copied, hacked or otherwise accessed in the electronic domain. Thus, hackers and other bad actors may not be able to electronically obtain the token or encryption key to access the electronic file stored in the service provider environment 102. In the printed configuration, the data file 116 can only be accessed with a printed copy of the token.

In an alternative configuration of the technology, the token or password encoded into the 2D optical code may be used to access the data store 118 but the token or password is not used to encrypt the data file itself. In this configuration, when a data file 116 is stored to a data store, the data file may be encrypted using an encryption key(s) managed by the data store 118. In order to access the data file 116 stored using the data store's encryption, a password is provided by the data store 118 to enable access to the data file in the data store 118 may be encoded into the 2D optical code. The password may be encrypted (e.g., 860 bit or higher) by the file router 110 or file service and may then be encoded in the 2D optical code. The 2D optical code can then be printed.

Later, the 2D optical code may be scanned in and the password in encrypted form may be sent from the client device 120 to the file router 110 or file service which decodes and decrypts the password and then sends the password to the data store 118. The password is then used to login to the data store 118. Once the login to the data store 118 has been validated, the data store can access the data file in the data store that was encrypted with a 256-bit or greater encryption key managed by the data store. The data store then decrypts and returns the data file to the client or another recipient using a data store managed encryption key. The password may alternatively be encrypted or decrypted by the data store 118 in some cases. Thus, the data store may encrypt or decrypt the password in another configuration. In addition, a client attribute or another user known term may also be provided with the encrypted password. This means the user may need to present the 2D optical code that the user has and also know something (e.g., the user data, user attributed or a code word).

One or more additional layers of security may also be applied to the optical code as the optical code passes through the network before printing and/or redemption. For instance, the optical code with the token, password or encryption key or may be sent through an encrypted network connection, tunnel or session. For example, when the optical code with the encoded token is sent for printing, the optical code may pass through the network in an encrypted state to prevent access to the optical code before the optical code is printed. More specifically, an encrypted connection (e.g., HTTPS, VPN (a virtual private network), TLS (transport layer security), etc.) can be created between the data store and file router, between the file router and the printer, between the file router and an application on a client (e.g., a smart phone), or between any other network computing links during network transport. In a further example, a user may receive a link through SMS texting. When the link is clicked on, a secure application may be launched on a mobile device or a secure browser connection may be created which securely communicates the optical code through the secure application or secure browser connection to the client.

As discussed, the token may be an encryption key. The data store may generate an encryption key and encrypt the data file with the encryption key. The encryption key may be a symmetric encryption key, an asymmetric encryption key, a public key, or a private key. For example, the token or encryption key may be a limited use token or encryption key that is a 256-bits, 860-bits, or any other length string or number of bits with a large number of possible characters per character position (e.g., with 62 or more character types).

In one configuration, an optical code may be used to store a default password for a network appliance or another internet enabled device. For instance, many networkable devices, such as routers, gateways, cameras, home automation devices, power generators, sensors, factory equipment and other internet enabled devices may have a default password and the default password is the same for every device manufactured of that type. Such devices have been easy to hack because thousands or millions of devices may use the same default password. Instead setting the same default administrator password for all the manufactured devices, each device may be provided with a unique and secure password that is 256-bits or more in length and this password may be encoded in a 2D optical code that is printed and shipped with the device or printed directly on the device. When a user wants to login to the device (e.g., login to perform admin functions on a camera), the 2D optical code may be scanned (e.g., by the camera or a mobile device) and administrative functions may be made available on the device. Thus, an encrypted login, password or an encryption key can be provided to a user using an optical code in a printed format or the device password can be retrieved from a secure data store using the token in the printed optical code. In addition, there will only be one physical copy of the 2D optical code and token as discussed before and no electronic copies. This makes the optical code difficult to hack.

In one example, the optical code with the token embedded can be used to transfer cash. For example, the technology may be used for cash transfers between individuals or companies. In addition, the printed optical code may be used for point of sale (POS) monetary exchange where the optical code may be scanned by a POS device to pay for a transaction. The token can be decoded from the optical code and then used to access financial information or account information stored in the cloud to complete the transaction.

The optical code may also be printed onto a paper check, as mentioned earlier. When the check with the optical code is presented for payment, then the optical code may be scanned and the token or digital code may be decoded from the optical code. This token can be used to check the authenticity of the check, since there is only one copy of the printed optical code. If a hacker or malicious party wanted to counterfeit the check, the hacker would need to have the token, password or encryption key embedded in the optical code. When the check is to be paid, the optical code can be scanned and the token, password, or encryption key can be decoded from the optical code. The token can be sent to the data store and secure information can be retrieved from the data file and the data file may provide secure data to validate and/or complete the transaction. Since the optical code has already been erased from any electronic storage medium, the optical code may not be copied electronically. In this scenario, printing a check that has a forged signature or other forged information but does not have the optical code will result in the check failing or being invalidated early in the check payment process (not days or weeks later as is currently the case).

A government entity may also want to transfer sensitive information between government employees or operatives in a secure way where the information is stored in data files located in centralized private or public data store. The government entity may store the secure data files (e.g., technology blue prints, maps, identities of individuals) in the data store where they are encrypted. Then the government entity may print a 2D optical code with the token, password or encryption onto paper or plastic. The printed optical code with the token, password or encryption key may be used to access that data file by scanning in the 2D optical code as discussed earlier. The only government employee who may access that file is the individual who has the printed optical code. This technology allows data to be transferred more easily and as securely as other highly secure data protection schemes.

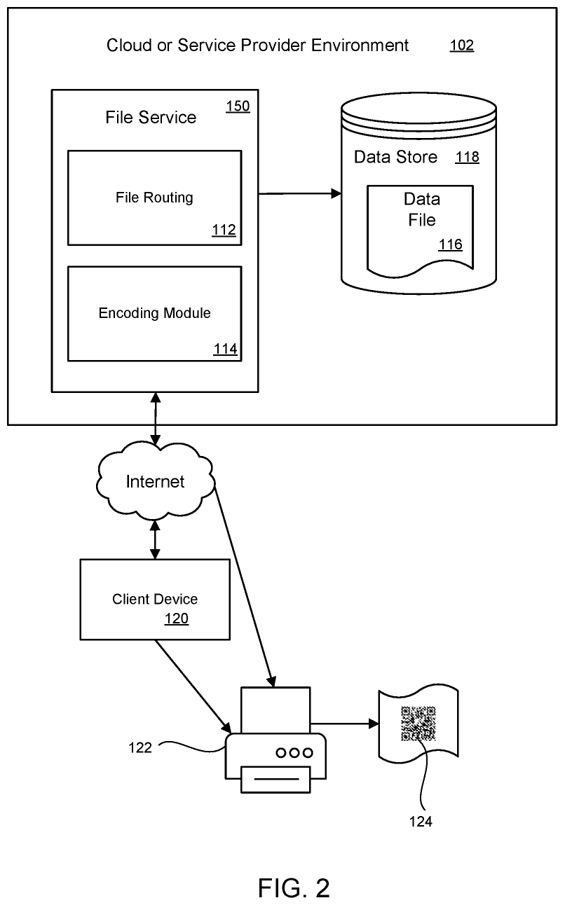

FIG. 2 illustrates technology to enable secure access to a protected or encrypted data file stored in a centralized data store or virtualized data store (e.g., in a private or public cloud) using a printed optical code provided to a user. Instead of using the file router of FIG. 1, a file service 150 may be used that is a virtualized service executing in a service provider environment 102 in a virtual machine, a container or a computing code function (e.g., Amazon Lambda). The file service 150 may receive a request to store a data file and operate using the same functions as the previously described file router. However, the file service 150 will be operating within the cloud as opposed to on a separate hardware router at the border of a local computer network to which the client is attached.

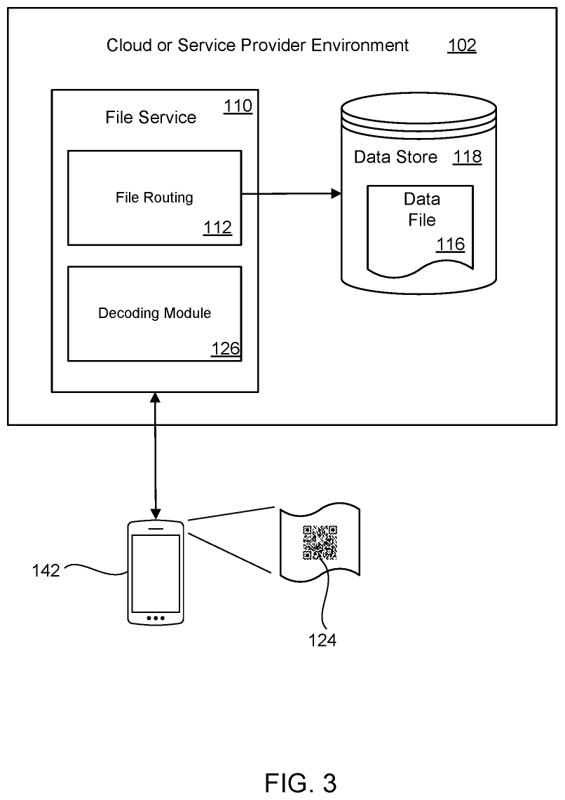

FIG. 3 illustrates making a request for and obtaining an encrypted data file 116 (e.g. an image file) using an encryption key that is encoded in a 2D barcode and physically printed. A 2D barcode or QR code can be scanned in using an optical scanner or an optical camera. For example, the camera of a mobile device 142, tablet, or personal computer may be used to scan in or capture the optical code 124 from a printed piece of paper or plastic, which was previously printed by a printer attached to another client device. The 2D optical code can be sent to a decoding module 126 which can decode a token, encryption key or password from the 2D barcode. Any additional encryption or decryption of passwords or tokens may also take place in the decoding module 126.

The file routing module 112 can send a request for the encrypted data file to the data store 118 of a service provider environment 102. The request to the data store 118 may include the token or encryption key decoded from the 2D barcode. The mobile device 142 or client may then receive the data file 116 that was decrypted by the data store using the encryption key. For example, the data file 116 may be a medical image that is displayed to a patient or medical professional who scanned the 2D barcode. The image file might also be a photo, computer rendering, CAD file, or another image file.

In one configuration, the token from the optical code may be decoded at the client device and then sent to the file service or file router. Then the file service can send the token or encryption key to the data store 118. As described before, the data store 118 can respond to the file service 150 with the decrypted data file in response receiving the token.

In another configuration, a user attribute may be associated with and also stored with the data file. The user attribute may be a user name, a user address, a user social security number, a user mobile phone number; a user identifier, or another user known value. The user attribute may be encrypted with the file or stored in plain text along with the file. If the 2D optical code has been received electronically, then a user attribute may be required for accessing the data file 116. If the 2D optical code was scanned in from a physical medium, then the user attribute may not be required.

The current technology enables users to be able transfer medical images quickly, easily and without remembering and typing in difficult codes. For example, QR codes are easily readable or scannable by a smart phone, a mobile device or another computing device with a camera or scanner. Even if a user were to receive an encryption key or token in a user readable format, users do not want to type in a very long code to obtain access to a data file because entering a code by hand is a cumbersome, slow, laborious and error prone process. In the case of this technology, the token or encryption key can be machine scanned in a fraction of a second.

As mentioned, a user may use the widely available technology of mobile devices with cameras to scan in the 2D optical code and access data files using web protocols. For example, mobile phones, tablets, laptops and other devices with cameras and scanners may be used to scan in the 2D optical code. Handheld scanners that are wired or wirelessly connected to a POS device, a computer or a computer network can also be used for scanning the 2D optical code. Then the data file may be delivered to the user using a web page or a mobile application.

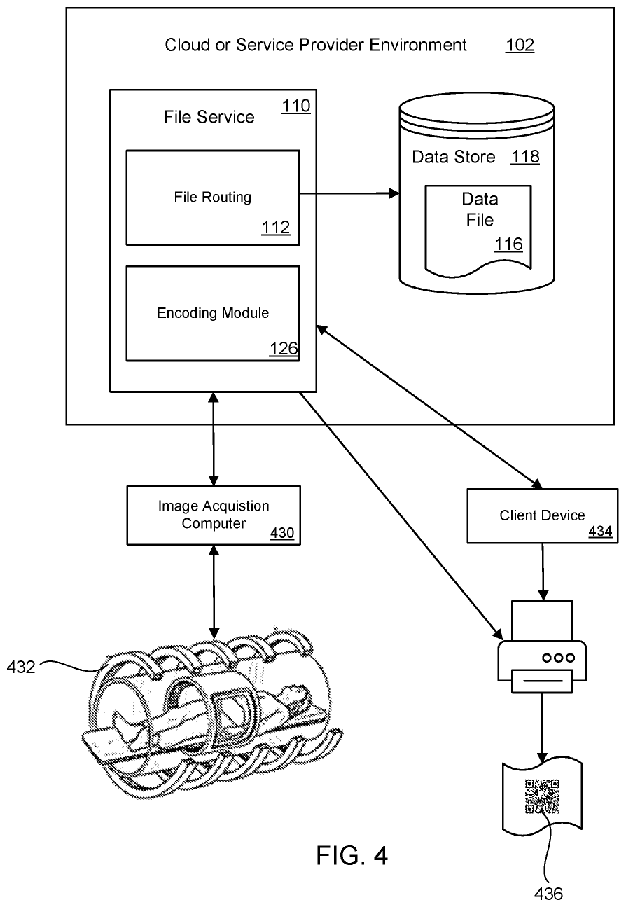

FIG. 4 illustrates a file storage system to store an image file (e.g., medical images or photographic images) that is accessible using an encryption key encoded and printed in a 2D optical code (e.g., QR code). Initially, an image file may be received from an image acquisition computer 430 over a computer network. The image acquisition computer may be in communication with medical imaging equipment 432 that enables medical professionals to capture an image file or data file for at least one medical image modality. Examples of medical image modalities that may be captured may include: an MRI (magnetic resonance imaging) modality, a Computerized Tomography (CT) scan modality, an X-ray modality, a Positron Emission Tomography (PET) modality, an ultrasound modality, a fluorescence modality, an Infrared Thermography (IRT) modality, 3D Mammography, or a Single-Photon Emission Computed Tomography (SPECT) scan modality. In another example, image data files may be an image or image data set which includes a combination of two or more forms of non-optical imaging modality as listed above (e.g., two or more images combined together, combined segments of two or more non-optical images, a CT image fused with an MRI image, etc.).

The image file may be received by the file routing module 112 of the file service 150, and the image file may be sent to a data store 118 to be stored in a service provider environment 102 which is accessible over the internet. The data store 118 may generate a token, file password or an encryption key to encrypt the image file to be stored in the data store. The data file may then be encrypted using the encryption key prior to storage in the data store 118.

A token, password, or encryption key for the image file may be received back from the data store 118 by the file service 150. The token, password, or encryption key for the image file or medical image may be encoded into an optical code using an encoding module 114. The optical code may be a 2D (two-dimensional) optical code. The 2D optical code may be sent to a client device 434 with access to a printer which can print the 2D optical code 436 on a physical or tangible medium. Printing the 2D optical code onto paper or plastic may enable sharing of the medical image with other medical professionals, individuals, or third parties not involved in the creation or storage of the medical image. Any electronic copies of the encryption key may be erased.

The 2D optical code 436 on paper or plastic may be received from the printer device by a medical patient or a medical professional. For example, a medical professional may print the 2D optical code so that a medical patient or another medical professional may later access medical images, medical documents, medical files, or medical instructions that the medical professional may want to provide to the medical patient or others.

In another medical example, it is not atypical for two health care companies to need to share medical information for a patient. However, health care company A may not want a medical professional from healthcare company B logging into their computing network or systems. Allowing such access may create a security risk and/or a public relations risk. However, healthcare company A and healthcare company B often need to share medical records and images. For example, multiple doctors may desire to create a virtual conference to discuss a medical condition of a patient, such as a tumor. The present technology allows health care companies or health care professionals to easily, quickly and securely share a medical data file. In this sense, the present technology may be considered a neutral but protected sandbox for sharing or exchanging medical images, medical documents, and medical data.

This technology is much more efficient and cost effective than sharing printed films, CDs (compact disks) or DVDs (digital versatile disks), hard drives or flash memories, which have been used in the past to share large medical images but such sharing methods are unsecure, slow and cumbersome. The use of physical media can also create problems because the physical media has to be transported physically and cannot be shared easily or repeatedly from a cloud-based location. In addition, using simple passwords have generally been a security risk because most doctors will not use or remember long passwords and neither will patients. This technology provides solutions for such issues.

Another challenge that hospitals and health care providers face is that the use of a CD or DVD to transfer a data file or medical images raises the problem of introducing viruses into the health care provider's devices or computer network. When an optical drive or other removable storage medium is used, there is the opportunity for receiving a virus. In fact, a hacker might socially engineer a scenario where a medical professional runs a CD that has a virus as part of medical treatment. However, the hospitals may need medical images that are vital to a patient's treatment. The present technology provides medical images to any doctor or medical institution that needs the images while avoiding the introduction viruses because the software for loading the medical image and the medical images come from trusted sources and do not use removable media.

In one configuration of this technology, if a user or creator of a data file shares the token or key electronically and the recipient does not have the physical code, then the recipient of the token or key may also need a person's attribute, which is associated with the creation of the file (e.g., a name of a patient who was medically imaged). So, if a doctor scans an electronic copy of the 2D optical code with the token, then the application may open a web page to provide the image about the patient but the doctor would also need to enter the patient's name, a patient's birthdate, a social security number, an address, a user's name, a challenge word, a code word, or another user attribute to access the data file or medical image.

If the user or recipient has the physical encryption key in their possession, then the user or recipient may not be asked to enter a name or user attribute. The physical code may have an additional encoding that indicates the 2D barcode is a physical printed copy and no other copies of the token exist. This additional coding in the 2D barcode which represents that the 2D barcode is a printed copy may indicate that a user attribute is not needed.

In an alternative configuration of the technology, a user attribute may be required each time a data file or image file is accessed. The user who wishes to access the data file must have the printed optical code and will be asked for a user attribute. This means the user needs to “have something” and “know something” to access the secure data file.

The present technology enables businesses or medical offices to share data files between businesses while avoiding cumbersome and lengthy security methods and/or additional computer hardware for setting up secure electronic connections or systems. For example, busy doctors may wish to share medical images with any other doctor in the United States but the size of the images, legal requirements of HIPPA, and balkanization of security computer systems may make that difficult. Sharing medical images using film, printed images or removable storage media (e.g., CD and DVDs) can also be cumbersome and undesirable. Furthermore, doctors do not want type in or remember large tokens, passwords, or other information to access medical information of a patient that has been referred to them for diagnosis or treatment. The present technology assists in addressing the problems discussed in a simple, efficient and secure way.

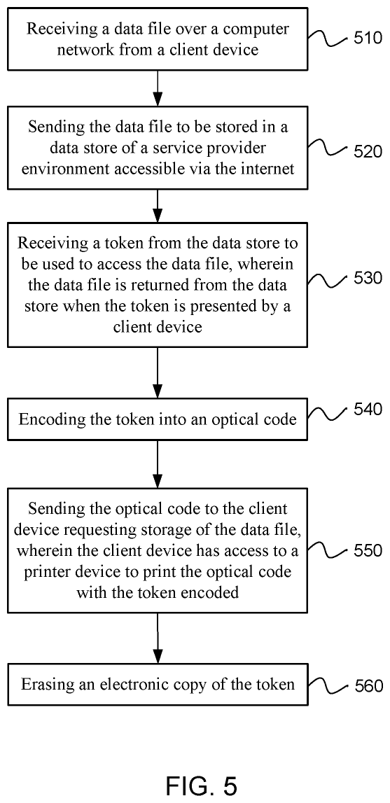

FIG. 5 illustrates a method for accessing a file using a printed optical code provided to user. A data file may be received over a computer network from a client device, as in block 510. The data file may be sent to be stored in a data store of a service provider environment accessible via the internet, as in block 520. Accordingly, this technology enables an image, such as a medical image, to be stored in an encrypted format in a data store in a public service provider environment (e.g., “cloud storage”) or private cloud storage. The image may be sent to a file router or service endpoint with a request to store the image on the data store in a service provider environment.

The file router can send back the token to client for the user who submitted the file or for a file that is associated with a user (e.g., a medical image of the user). A token may be received from the data store to be used to access and/or decrypt the data file, as in block 530. The data file can be returned from the data store when the token is presented by a client device or other device.

The token can be encoded into an optical code, as in block 540. For example, the optical code may be a 2D optical code and the token can be embedded into the 2D optical code. The optical code may be sent to the client device requesting storage of the data file, as in block 550. The client device may have access to a printer device to print the optical code with the token encoded. An electronic copy of the token may also be erased, as in block 560.

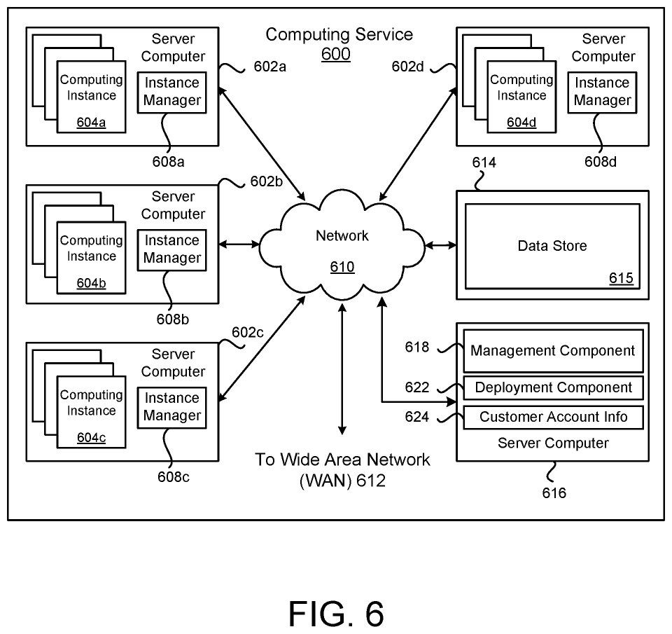

FIG. 6 is a block diagram that illustrates an example computing service 600 or service provider environment that includes virtualized processing and virtualized data file storage according to one example of the present technology. The computing service 600 may be used to execute and manage a number of computing instances 604a-d upon which the present technology may execute. In particular, the computing service 600 depicted illustrates one environment in which the technology described herein may be used. The computing service 600 may be one type of environment that includes various virtualized service resources that may be used, for instance, to host computing instances 504a-d.

The computing service 600 may be capable of delivery of computing, storage, and networking capacity as a software service to a community of end recipients. In one example, the computing service 600 may be established for an organization by or on behalf of the organization. That is, the computing service 600 may offer a “private cloud environment.” In another example, the computing service 600 may support a multi-tenant environment, wherein a plurality of customers may operate independently (i.e., a public cloud environment). Generally speaking, the computing service 600 may provide the following models: Infrastructure as a Service (“IaaS”), Platform as a Service (“PaaS”), and/or Software as a Service (“SaaS”). Other models may be provided. For the IaaS model, the computing service 600 may offer computers as physical or virtual machines and other resources. The virtual machines may be run as guests by a hypervisor, as described further below. The PaaS model delivers a computing platform that may include an operating system, programming language execution environment, database, and web server.

Application developers may develop and run their software solutions on the computing service platform without incurring the cost of buying and managing the underlying hardware and software. The SaaS model allows installation and operation of application software in the computing service 600. End customers may access the computing service 600 using networked client devices, such as desktop computers, laptops, tablets, smartphones, etc. running web browsers or other lightweight client applications, for example. Those familiar with the art will recognize that the computing service 600 may be described as a “cloud” environment.

The particularly illustrated computing service 600 may include a plurality of server computers 602a-d. The server computers 602a-d may also be known as physical hosts. While four server computers are shown, any number may be used, and large data centers may include thousands of server computers. The computing service 600 may provide computing resources for executing computing instances 604a-d. Computing instances 604a-d may, for example, be virtual machines. A virtual machine may be an instance of a software implementation of a machine (i.e. a computer) that executes applications like a physical machine. In the example of a virtual machine, each of the server computers 602a-d may be configured to execute an instance manager 608a-d capable of executing the instances. The instance manager 608a-d may be a hypervisor, virtual machine manager (VMM), or another type of program configured to enable the execution of multiple computing instances 604a-d on a single server. Additionally, each of the computing instances 604a-d may be configured to execute one or more applications.

A server computer 614 may be reserved to execute software components for implementing the present technology. For example, the server computer 614 may execute at least a portion of virtualized data store 615 to store data files that have been encrypted or password protected. Another server computer 602a-d may be used as a file service for encrypting data files and/or routing the data files to the data store 615.

A server computer 616 may execute a management component 618. A customer may access the management component 618 to configure various aspects of the operation of the computing instances 604a-d purchased by a customer. For example, the customer may setup computing instances 604a-d and make changes to the configuration of the computing instances 604a-d.

A deployment component 622 may be used to assist customers in the deployment of computing instances 604a-d. The deployment component 622 may have access to account information associated with the computing instances 604a-d, such as the name of an owner of the account, country of the owner, etc. The deployment component 622 may receive a configuration from a customer that includes data describing how computing instances 604a-d may be configured. For example, the configuration may include an operating system, provide one or more applications to be installed in computing instances 604a-d, provide scripts and/or other types of code to be executed for configuring computing instances 604a-d, provide cache logic specifying how an application cache is to be prepared, and other types of information. The deployment component 622 may utilize the customer-provided configuration and cache logic to configure, prime, and launch computing instances 604a-d. The configuration, cache logic, and other information may be specified by a customer accessing the management component 618 or by providing this information directly to the deployment component 622.

Customer account information 624 may include any desired information associated with a customer of the multi-tenant environment. For example, the customer account information may include a unique identifier for a customer, a customer address, billing information, licensing information, customization parameters for launching instances, scheduling information, etc. As described above, the customer account information 624 may also include security information used in encryption of asynchronous responses to API requests. By “asynchronous” it is meant that the API response may be made at any time after the initial request and with a different network connection.

A network 610 may be utilized to interconnect the computing service 600 and the server computers 602a-d, 616. The network 610 may be a local area network (LAN) and may be connected to a Wide Area Network (WAN) 612 or the Internet, so that end customers may access the computing service 600. In addition, the network 610 may include a virtual network overlaid on the physical network to provide communications between the server computers 602a-d. The network topology illustrated has been simplified, as many more networks and networking devices may be utilized to interconnect the various computing systems disclosed herein.

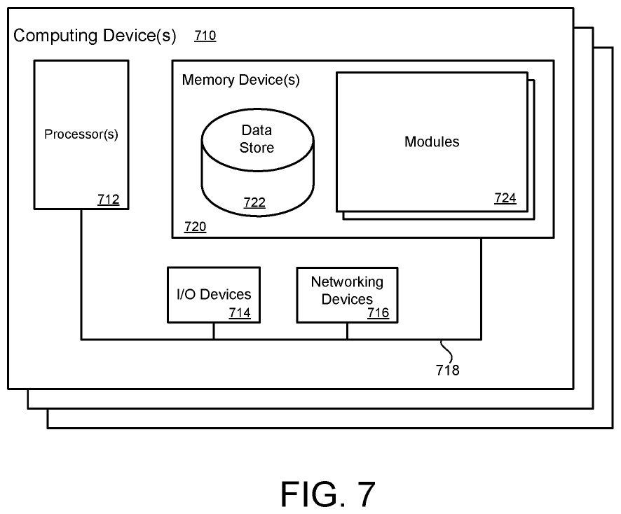

FIG. 7 illustrates one or more computing device(s) 710 on which modules or code components of this technology may execute. A first computing device 710 is illustrated on which a high-level example of the technology may be executed. The first computing device 710 may include one or more processor(s) 712 that are in communication with memory device(s) 720. The computing device may include a local communication interface 718 for the components in the computing device. For example, the local communication interface may be a local data bus and/or any related address or control busses as may be desired.

The memory device(s) 720 may contain modules 724 or code components that are executable by the processor(s) 712 and data for the modules 724. The modules 724 may execute the functions described earlier. A data store 722 may also be located in the memory device(s) 720 for storing data related to the modules 724 and other applications along with an operating system that is executable by the processor(s) 712.

Other applications may also be stored in the memory device(s) 720 and may be executable by the processor(s) 712. Components or modules discussed in this description that may be implemented in the form of software using high programming level languages that are compiled, interpreted, or executed using a hybrid of the methods.

The computing device may also have access to I/O (input/output) devices 714 that are usable by the computing devices. An example of an I/O device is a display screen that is available to display output from the computing devices. Other known I/O device may be used with the computing device as desired. The networking devices 716 and similar communication devices may be included in the computing device. The networking devices 716 may be wired or wireless networking devices that connect to the internet, a LAN, WAN, or other computing network.

The components or modules that are shown as being stored in the memory device(s) 720 may be executed by the processor(s) 712. The term “executable” may mean a program file that is in a form that may be executed by a processor(s) 712. For example, a program in a higher-level language may be compiled into machine code in a format that may be loaded into a random-access portion of the memory device(s) 720 and executed by the processor(s) 712, or source code may be loaded by another executable program and interpreted to generate instructions in a random-access portion of the memory to be executed by a processor. The executable program may be stored in any portion or component of the memory device(s) 720. For example, the memory device(s) 720 may be random access memory (RAM), read only memory (ROM), flash memory, a solid-state drive, memory card, a hard drive, optical disk, floppy disk, magnetic tape, or any other memory components.

The processor(s) 712 may represent multiple processors and the memory 720 may represent multiple memory units that operate in parallel to the processing circuits. This may provide parallel processing channels for the processes and data in the system. The local communication interface 718 may be used as a network to facilitate communication between any of the multiple processors and multiple memories. The local communication interface 718 may use additional systems designed for coordinating communication such as load balancing, bulk data transfer, and similar systems.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A method to store a data file that is accessible using a token encoded in a printed optical code, comprising:
receiving the data file over a computer network from a client device requesting secure storage of the data file;
sending the data file to be stored in a data store of virtualized data storage accessible via a public network;
receiving a token from the data store to be used to access the data file, wherein the data file is returned from the data store when the token is received from the client device;
encoding the token into an optical code;
sending the optical code to the client device requesting storage of the data file, wherein the client device has access to a printer device to print the optical code with the token encoded; and
erasing an electronic copy of the token.

2. The method as in claim 1, further comprising printing the optical code with the token encoded.

3. The method as in claim 2, further comprising printing an optical code that is a 2D optical code with the token on paper or plastic.

4. The method as in claim 3, further comprising supplying the 2D optical code on paper to a medical patient or a medical professional.

5. The method as in claim 1, wherein the optical code is at least one of: a 2D barcode, a QR code, a PDF 417 code, an Aztec barcode, or a one-dimensional barcode.

6. The method as in claim 1, wherein the data file is a medical image.

7. The method as in claim 3, further comprising:
scanning the optical code that was previously printed, at the client device;
decoding the token from the optical code;
sending the token to the data store; and
receiving the data file that is decrypted from the data store in response supplying the token to the data store.

8. The method as in claim 1, further comprising associating and storing a user attribute with the data file.

9. The method as in claim 8, wherein the user attribute is at least one of: a user name, a user address, a user social security number, a user mobile phone number, a user identifier, or a user known value.

10. The method as in claim 8, further comprising:
scanning the optical code that has been printed;
decoding the token from the optical code;
sending the token and the user attribute to the data store; and
receiving the data file in response to sending the token.

11. The method as in claim 1, wherein the token is an encryption key.

12. The method as in claim 11, wherein the encryption key is at least one of: a symmetric encryption key, an asymmetric encryption key, a public key, or a private key.

13. The method as in claim 1, wherein a token and a URL (uniform resource locator) which represents a data store where the data files stored are encrypted and encoded into the optical code.

14. A system to store an image file that is accessible using an encryption code printed in a 2D optical code, comprising:
one or more processors; and
a memory storing instructions which, when executed by the one or more processors, cause the one or more processors to:
receive an image file, over a computer network, from a client device requesting secure storage of the image file;
send the image file to be stored in a data store of a service provider environment accessible over a public network;
receiving an encryption key, from the data store, for the image file which has been encrypted with the encryption key;
encoding the encryption key for the image file into a 2D (two dimensional) optical code;
sending the 2D optical code to a client device with access to a printer to print the 2D optical code; and
erasing an electronic copy of the encryption key for the image file.

15. The system as in claim 14, further comprising printing the 2D optical code on paper or plastic using a printer.

16. The system as in claim 14, wherein the image file is a medical image.

17. The system as in claim 14, further comprising providing the 2D optical code on paper to a medical patient or a medical professional to enable sharing of a medical image with third parties.

18. The system as in claim 14, wherein the image file is at least one of an MRI (magnetic resonance image) file, a CT (computed tomography) image file, or an ultrasound image file.

19. The system as in claim 14, wherein the encryption key is at least one of a symmetric encryption key, an asymmetric encryption key, or a private encryption key that is part of a public-private encryption key pair.

20. A method for obtaining an encrypted image file using an encryption key that is printed in a 2D barcode, comprising:
scanning the 2D barcode using an optical scanner of a computing device;
decoding the encryption key from the 2D barcode;
sending a request for an image file to a file router that is in communication with a data store of a service provider environment, wherein the request includes the encryption key decoded from the 2D barcode and a user attribute that is checked by the data store; and
receiving the image file that was decrypted using the encryption key from the data store.

21. The method as in claim 20, wherein a medical image in the image that was decrypted is displayed to a patient or medical professional who scanned the 2D barcode.

22. A method for providing a token to a user for use in accessing protected data, comprising:
generating a token used to login to a secure resource accessible via a computing environment;
encoding the token into an optical code;
printing the optical code onto a printable medium; and
providing the optical code to a user who desires to access the secure resource accessible via the computing environment.

23. The method as in claim 22, further comprising:
scanning the optical code using an optical scanner of a computing device;
decoding the token from the optical code in the computing device; and
applying the token in the computing device to access the secure resource.

24. The method as in claim 22, further comprising:
scanning the optical code using an optical scanner of a computing device;
decoding the token from the optical code in the computing device; and
applying the token in the computing device to access a secure web site or web application.

25. The method as in claim 22, further comprising:
scanning the optical code using an optical scanner of a computing device;
sending a request for a data file to a file router that is in communication with a data store of a service provider environment, wherein the request includes the token decoded from the optical code;
receiving the data file that was decrypted from the data store; and
applying a login and password, at the computing device from the data file to a secure website or web application.

26. The method as in claim 22, wherein the token is a password, login, or encryption key.

27. The method as in claim 26, wherein the token is a login or password to an electronic device.

28. A method to store a data file that is accessible using a password encoded into a printed optical code, comprising:
receiving the data file over a computer network from a client device requesting secure storage of the data file;
encrypting the data file using an encryption key managed by a data store of virtualized data store service accessible via a public network;
storing the data file in the data store;
sending a password from the data store to be used to access the data file, wherein the data file is to be returned from the data store when the password is received from the client device; and
erasing an electronic copy of the password at the data store.

29. The method as in claim 28, further comprising:
encrypting the password;
encoding the password into an optical code; and
sending the optical code with the password encoded to a printer device to print the optical code.
